(19) United States
(12) Patent Application Publication
(10) Pub. No.: US 2007/0123799 A1
(43) Pub. Date: May 31, 2007

(54) Method and kit for biopsying of pancreatic tumor masses

(75) Inventor: Ozanan R. Meireles, East Lansing, MI (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 11/582,184

(22) Filed: Oct. 17, 2006

Related U.S. Application Data
(60) Provisional application No. 60/727,307, filed on Oct. 17, 2005.

(51) Int. Cl. A61B 10/00

(52) U.S. Cl. 600/564

(57) ABSTRACT

A method and kit for biopsying of the pancreas is disclosed. The biopsy is laparoscopic with a gastroscope. The method is more economic and safer than standard procedures.

### CROSS-REFERENCE TO RELATED APPLICATIONS

This application is based for priority on U.S. Provisional Application Ser. No. 60/727,307 filed Oct. 17, 2005.

### STATEMENT REGARDING FEDERALLY SPONSORED RESEARCH OR DEVELOPMENT

Not Applicable

### STATEMENT REGARDING GOVERNMENT RIGHTS

Not Applicable

### BACKGROUND OF THE INVENTION

(1) Field of the Invention

The present invention relates to a method and kit for biopsying of pancreatic tumor masses using a unique dimensional, flexible laparoscopic procedure with a gastroscope. The procedure is rapid, economic and relatively non-invasive.

(2) Description of Related Art

Symptomatic pancreatic masses have low prevalence in the United States; however, as cross-sectional imaging has improved and become more widely used in aging populations, those tumors are now encountered as an incidental finding with greater frequency.

For the determination of the nature of many pancreatic masses a final diagnosis, histologic or cytologic confirmation, is almost always needed for depicting the treatment plan. The reported sensitivity and diagnostic accuracy are high for both histologic and cytologic examinations. Specificity is near to 100% in most published series, from centers of excellence. However, in practice, obtaining a diagnosis by means of percutaneous biopsy can be challenging.

Percutaneous Fine Needle Aspiration (FNA) biopsy is a modality of tissue sampling already in use. In this technique, the needle is guided by Ultrasound (US) or Computer Aided Tomography (CT or CAT) toward the tissue that will be sampled, but has a potential risk of bowel and vascular injuries, besides posing a small but real risk of tumor implantation along the biopsy track of the needle.

The sensitivity of US-guided FNA is 75-97%, similar to that of CT-guided FNA. Similar high specificity is found with US-guided FNA of lymph nodes. An FNA specimen is almost always adequate.

The Transduodenal Endoscopic Ultrasound guided biopsy has some advantages pertaining to the approach, avoiding the biopsy track tumor implantation and reduces pancreatic fistula formation. But comparing with FNA, the Tranduodenal approach using Endoluminal Endoscopic Ultrasound is limited to the head of the pancreas.

Other options are Open or Standard Laparoscopic Pancreatic biopsy, which incur in a bigger surgical procedure, requiring in some instances long operating room time, complex instruments and hospital stay, without mention of the morbidity of the surgical stress.

Symptomatic pancreatic masses have a low prevalence in the United States; however, as cross-sectional imaging has improved and become more widely available in an aging population, those tumors are now encountered as an incidental finding with greater frequency. For the determination of their nature and for outlining the treatment plan, histologic or cytologic confirmation is almost always needed. The reported sensitivity and diagnostic accuracy are high for both histologic and cytologic examinations and their specificity is near to 100% in most published series. Even so, in practice, obtaining a diagnosis by means of percutaneous biopsy can be challenging.

Some methods of tissue sampling currently being used show excellent success targeting masses located in the head of the pancreas, but from the farther it gets from the duodenum the chance of success diminishes and the risks increase, due to the pancreatic body and tail's anatomic location and relation to other structures. Because of this, many surgeons advocate more complex laparoscopic procedures, or even open procedures to rule out malignancy.

Previously, it has been demonstrated the value of laparoscopic assessment of the pancreas but limitations have been noted, especially due to instrumentation.

### OBJECTS

It is therefore an object of the present invention to provide a new procedure for the biopsy of tumor masses on the pancreas. It is further an object of the present invention to provide a procedure which is rapid, erosive and relatively non-invasive.

The substance and advantages of the present invention will become increasingly apparent by reference to the following drawings and the description.

### SUMMARY OF THE INVENTION

The present invention relates to a method for diagnosing pancreatic tumor masses in vivo in a patient which comprises:
(a) installing a laparoscopic port in an incision in the patient at about 10-12 mm at the right quadrant upper mid line of the patient;
(b) introducing a flexible gastroscope with a work channel with a length between about 8.6 and 12 mm through the port and into the peritoneal cavity of the patient with gas inflation of the peritoneum;
(c) introducing the gastroscope into the lesser sac in the peritoneum cavity and through the epiploic foramen adjacent to the pancreas;
(d) introducing biopsy forceps through the gastroscope work channel and removing a portion of a mass on the pancreas; and
(e) removing the endoscope from the patient and disinflating the peritoneal cavity, removing the laparoscopic port and closing the incision.

Further, the present invention relates to a method wherein the pancreas surgically enables repair of the pancreas.

Still further, the present invention relates to a kit for performing the above described method comprising the gastroscope with the work channel and biopsy forceps adapted for the method which are disposable, the port, and means for closing the incision.

### BRIEF DESCRIPTION OF FIGURES

The patent or application file contains at least one drawing executed in color. Copies of this patent with color drawings(s) will be provided by the Patent and Trademark Office upon request and payment of necessary fee.

FIG. 1 is a photograph showing the position of tissues shown in FIG. 1.

FIG. 2 is a schematic drawing of the organs in the peritenial cavity of a patient (human).

FIG. 3 is a photograph showing the Foramen of Winslow also known as the Epiploic Foramen which is adjacent to the pancreas and below the Foramen.

### DESCRIPTION OF THE PREFERRED EMBODIMENTS

Pancreatic tumors require histologic or cytologic confirmation for diagnosis and subsequent treatment. Currently available methods for pancreatic biopsy are limited and pose undesirable risks. Our group has demonstrated that Lesser Sac Flexible Peritoneoscopy for pancreatic evaluation and biopsy is feasible in an animal model. Methods: Using a canine model, a flexible gastroscope was introduced transabdominally and advanced through the epiploic foramen (Winslow) providing visualization and access to the pancreas and adjacent structures within the lesser sac. Pancreatic biopsy was performed and sent for histological confirmation. Results: Lesser Sac Flexible Peritoneoscopy was feasible in an animal model with a standard flexible gastroscope. The pancreas and surrounding organs were evaluated under direct vision with adequate picture quality. Endoscopic forceps (used for biopsy) provided sufficient pancreatic tissue, which was confirmed on post-mortem celiotomy. There were no complications during these procedures. Conclusions: Lesser Sac Flexible Peritoneoscopy is feasible in an animal model using a commercially-available gastroscope. This novel procedure is potentially less invasive than the standard laparoscopic approach to the lesser sac. When fully developed will have the potential to improve patient care by an increased success rate over current image guided biopsy methods for the evaluation of pancreatic masses.

A new, better, efficient, more accurate and less invasive procedure is provided and associated with less instrumentation and low cost. Conventional laparoscopic equipment is adapted to perform a unique procedure, never described before, but feasible with the current technology and machinery available, and at the same time with a considerable potential for development of new devices and tools that will improve patient care.

This procedure can be accomplished with the introduction of a conventional flexible gastroscope through a 10-12 mm laparoscopic port, and once inside the peritoneal cavity under insufflation with a gas, the gastroscope was advanced cranially in the right abdomen, dorsal to the hepatic artery and portal vein, and medially through the Epiploic Foramen (Winslow Foramen) providing anatomic access to the Lesser Sac. At this point, the pancreas was inspected under direct visualization and tissue sampling performed with the gastroscope biopsy forceps.

LSFP provides better access for Pancreatic Biopsy and represents a significant decrease in complications such as Pancreatic Fistula formation, compared with the other approaches, due to the direct visualization and due to the potential therapeutic procedures that can be performed (fibrin glue or clipping).

Again, Tissue Sampling has a clear advantage in patients with questionable tumor etiology in whom the diagnosis should preferably be established without subjecting the patient to surgery. The benefits are (1) efficiency in the pancreatic mass diagnosis; (2) more accuracy; (3) less invasiveness; (4) less instrumentation; (5) lower cost; (6) direct gross visualization; lower morbidity; and decreased cosmetic insult.

Our group has demonstrated that Lesser Sac Flexible Peritoneoscopy (LSFP) for pancreatic evaluation and biopsy is feasible in a canine model. This novel procedure, is less invasive than standard laparoscopic pancreatic biopsy, requires less instrumentation and is potentially more accurate for tissue sampling than current radiologic-assisted methods for selected tumors located in the pancreatic body or tail.

### Materials and Methods

All Procedures were reviewed and approved by the Michigan State University Institutional Animal Care and Use Committee. Two male canines averaging 13 Kg were housed in kennels and allowed access to standard chow and water ad libitum. Dogs were fasted for 16 h prior to the procedures. Both canines were under Isoflurane general anesthesia during the surgical procedures and subsequently euthanized with an intravenous overdose of barbiturate.

To assess the feasibility of the Transabdominal Lesser Sac Flexible Peritoneoscopy (LSFP) in the animal model, a group of 5 General Surgeons and 1 Veterinary Surgeon worked collaboratively during a course of several weeks.

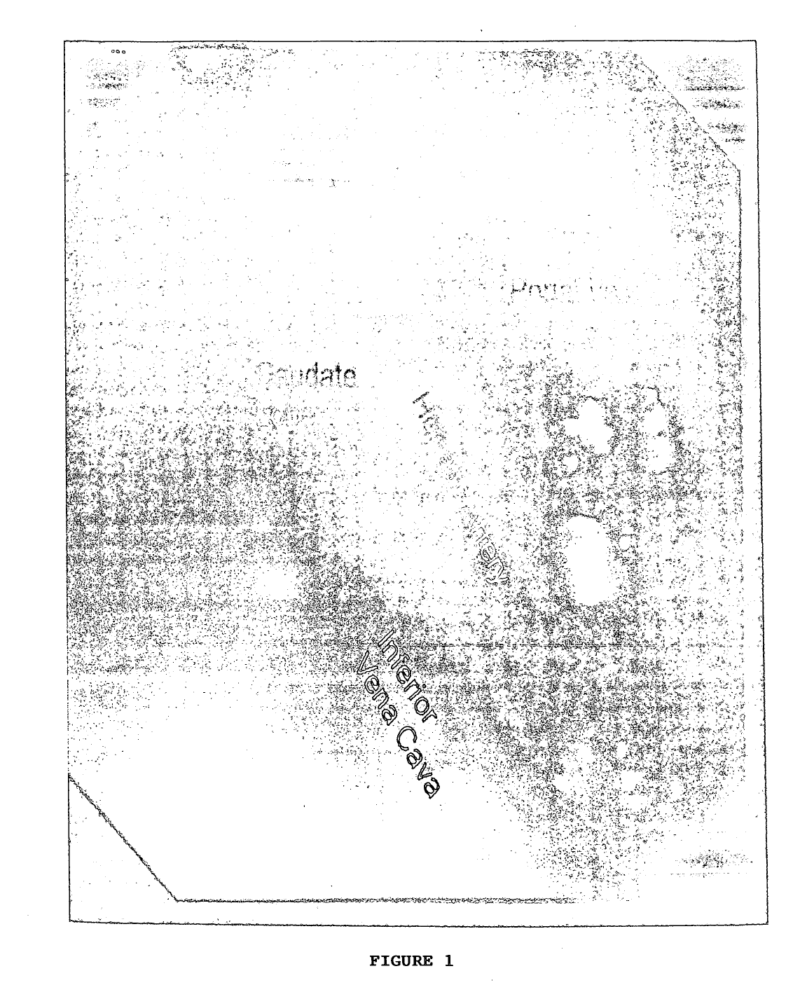

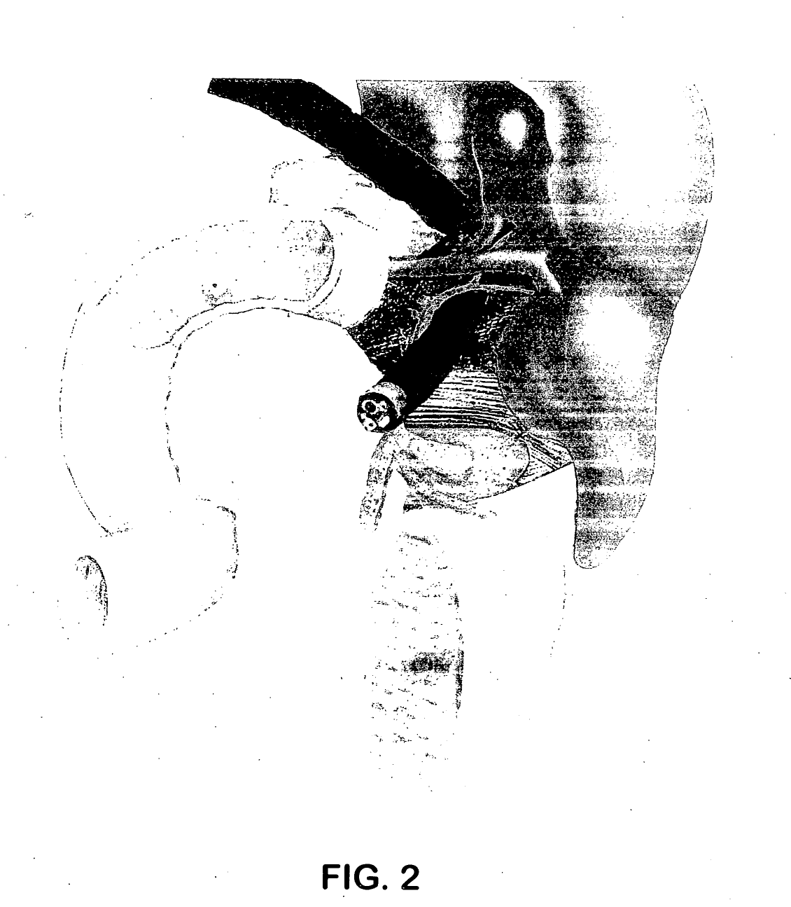

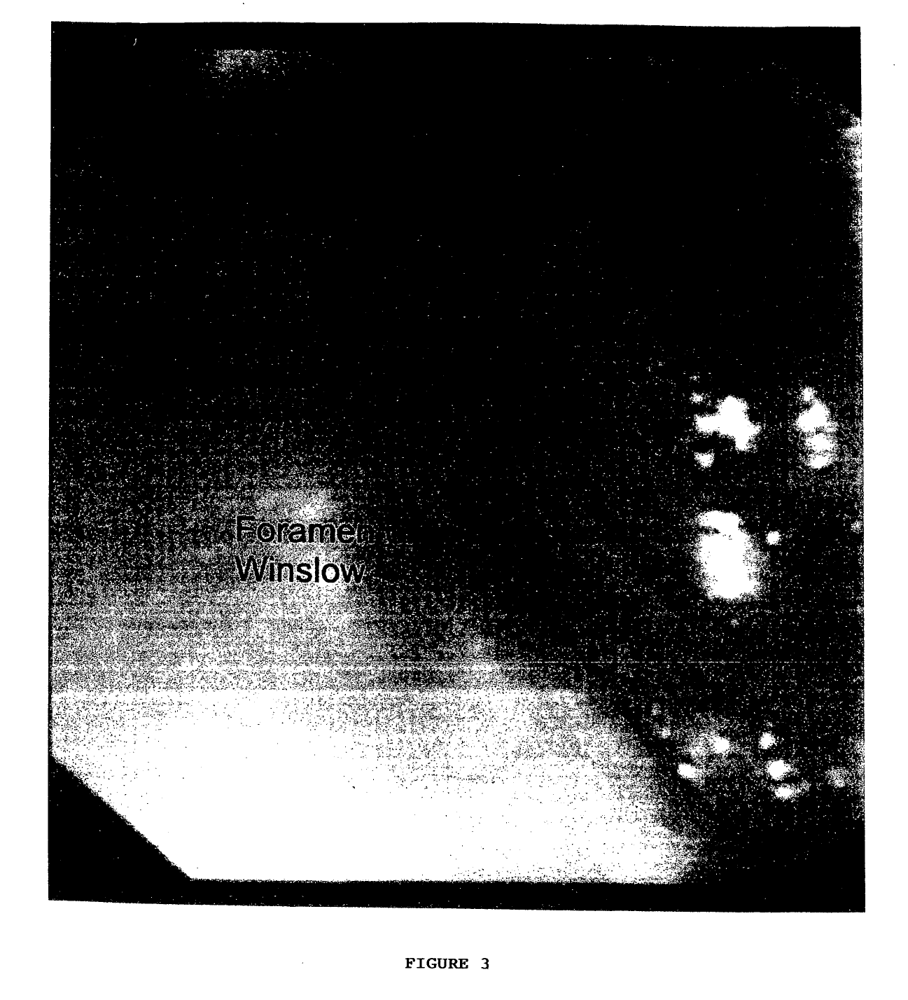

Under general anesthesia, an Olympus flexible gastroscope GIF-Q160, with 9.5 mm outer diameter insertion tube and 2.8 mm working channel diameter, was inserted into the abdomen through a supraumbelical 10 mm laparoscopic port after pneumoperitoneum achieved with Veress needle. Laparoscopy was performed and under direct visualization a second port was placed in the RUQ near the tip of the right 12^{th} rib, allowing the placement of a laparoscopic retractor through the epiploic foramen (“Foramen of Winslow”—FIGS. 1 to 3) for gastric retraction and pancreatic exposure. With the gastroscope in position, the laparoscopic retractor and the gastroscope were advanced under direct visualization through the epiploic foramen (Winslow) providing anatomic access to the lesser sac. The posterior gastric wall was retracted anteriorly with a Babcock retractor concomitant with continuous CO2 insufflation via the gastroscope allowing the lesser sac to expand from its virtual space to a segmental pneumoperitoneum. The gastroscope was maneuvered throughout the lesser sac providing visualization and access to the anterior surfaces of the pancreas neck, body, and tail and adjacent structures within the lesser sac (FIG. 2). The foramen is shown in FIG. 3.

Under clear visualization of the anterior surface of the pancreas, a Biopsy Forceps—Alligator Jaw-Step with Cup Opening of 7.2 mm was used through the working channel to perform pancreatic biopsy, with an average of two attempts to obtain adequate tissue, which was sent to pathology for histological confirmation. After the biopsy had been performed the biopsied site was observed for 1 minute to identify bleeding or pancreatic leak. At the end of the procedure the dogs were euthanized under general anesthesia followed by open necropsy, to visualize the actual biopsy site.

### Results

Both dogs remained hemodynamically stable throughout the procedure and were maintained continuously under gene anesthesia. There were no complications.

On both procedures we were able to enter the lesser sac with flexible gastroscope and guide the laparoscopic retractor without any problems. Once inside the lesser sac, the gastroscope insuflation was insufficient to provide full expansion of the lesser sac compartment due to lack of seal at the foramen of Winslow level, where CO2 was escaping into the greater sac, but with the utilization of a laparoscopic retractor retracting the posterior aspect of the portal vein anteriorly, the anterior surface of the pancreas became much more accessible to the gastroscope camera, allowing us to better discriminate anatomic structures and their relations within the lesser sac. A sealing ring can be provided on the gastroscope to seal the foramen.

The pancreatic biopsy was easily obtained on each dog under endoscopic visualization of the pancreas and the biopsy sites were confirmed to be pancreas on post-mortem celiotomy with direct inspection.

### Discussion

Pancreatic tumors account for a fair number of mortalities every year in this the United State, estimated at approximately 20,000. While the mortality of surgical treatment of these malignancies has fallen substantially with improvements in surgical and post-operative care, overall mortality has not. One of the primary reasons is delay in diagnosis. Pancreatic malignancies are notorious for their late presentation—particularly when located in the body and tail. The clinical diagnosis is usually made when advanced disease is present with pronounced clinical symptoms, but a growing number of pancreatic masses are being diagnosed in their early and stages due increased use of high quality imaging studies in the general population, especially in the elderly, as incidental findings from routine tests or from other reasons non-related to the pancreas that required imaging of the abdomen.

A large percentage of those patients are asymptomatic at the time of the diagnosis, but because of the ominous nature of pancreatic cancer, aside with a variety of benign and malignant neoplasms, the histological diagnosis is paramount in guiding the appropriate therapy.

A distinction should be made regarding the actual location of the abnormality within the pancreas: head versus body and tail, and anterior aspect versus posterior aspect. It is important to emphasize that although current biopsy methods may show high success rates in obtaining tissue for cytology or histology for masses located in the head of the pancreas, they may also present some limitations and undesirable risks. It also seems that the further a pancreatic abnormality gets from the duodenum the greater the risk of complications.

Computed tomography-guided percutaneous fine-needle aspiration (CT-FNA) is the biopsy procedure of choice for pancreatic mass lesions at many institutions, however this method has a significant false-negative rate, close to 20%. Also the needle has the potential of tract seeding after percutaneous biopsies.

Endoscopic ultrasonography-guided Fine Needle Aspiration biopsy (EUS-FNAB) of the pancreas is well accepted and some times is more accurate than CT-FNA, showing sensitivity raging from 80%-94.7%, specificity 82%-100% and positive predictive value close to 100% for solid pancreatic masses located in the pancreatic head. Although EUS-FNAB presents higher accuracy, it is confined to the pancreatic head, therefore not being able to reach the mid and distal pancreas.

The laparoscopic approach provides direct visualization, thus enabling optimal direct tissue sampling, but requires multiple port placements along with more complex tissue manipulation and dissection.

Our group envisioned and successfully performed the first Transabdominal Lesser Sac Flexible Peritoneoscopy with pancreatic biopsy. The LSFP is a novel technique that merges concepts from laparoscopy and endoluminal flexible endoscopy. As described in detail above, a standard flexible gastroscope enters the peritoneal cavity through a regular laparoscopic port and then gains anatomic access to the lesser sac cavity via the foramen of Winslow, without requiring any tissue dissection. From this point, the pancreas can be biopsied under direct visualization using a biopsy forceps through the scope's working channel. This technique requires a minimal abdominal incision.

The procedure time was greater on the first experiment, as expected, due to the different anatomy of the canine, and with the learning curve necessary to operate a flexible gastroscope freely into the peritoneal cavity. Anatomic differences were outlined by the veterinary surgeon (BJS), and adjustments in technique were easily undertaken as our team became more comfortable with the use of the gastroscope in this setting.

LSFP provides critical benefits over other methods, since this technique allows the biopsy forceps to reach the body and tail of the pancreas without being impeded by other organs, therefore avoiding the potential risks and complications of CT-FNA and going beyond EUS-FNAB range.

Some tangible disadvantages of LSFP at this current state of development need to be discussed, such as the lack of ability to assess the posterior aspect of the pancreas. Nevertheless this could be resolved with the addition of US capabilities to the gastroscope, or with further modification and refinement of the technique. Even with this limitation, LSFP still an important step, since before it, access to the anterior aspect of the body and tail of the pancreas was limited.

The fact that a second port was necessary to avoid the lesser sac to collapse into its virtual space, may sound less appealing than the use of a single port with a yet conceptual multifunctional flexible scope. But the pilot nature of this project, however, was to prove procedural feasibility. Our ultimate goal, and we have some projects ongoing on this regard, is to provide a new scope apparatus capable of providing a seal at the foramen of Winslow level and maintain a compartmental pneumoperitoneum within the lesser sac space.

### Conclusion

In conclusion, LSFP is feasible in an animal model with a commercially-available gastroscope. This procedure combines laparoscopic and endoscopic techniques and represents a novel method of minimally invasive access to the lesser sac. With this technique, the body and tail of the pancreas can be biopsied under direct visualization using a minimally invasive access and without tissue dissection. In this pilot study for feasibility, no complications were encountered during the procedure, including iatrogenic damage to other structures.

It is clear that further studies are necessary before human application is attempted, focusing in different areas regarding physiological changes, potential procedural complications, biopsy accuracy, development of new equipment and translational studies. LSFP has the potential to improve patient care, minimize surgical trauma and optimize the timeframe and accuracy on the diagnosis of mid and distal pancreatic masses going beyond the reach of current CT and EUS-guided biopsy methods.

### REFERENCES

1. Landis S H, Murray T, Bolden S, Wingo P A. Cancer statistics, 1998. C A Cancer J Clin. 1998; 48:6-29.
2. Jemal A, Tiwari R C, Murray T, et al. Cancer statistics, 2004. CA Cancer J Clin. 2004; 54:8-29.
3. Sheehan M, Latona C, Aranha G, Pickleman J. The increasing problem of unusual pancreatic tumors. Arch Surg. 2000; 135: 644-8.
4. Rattner D W, Fernandez-del Castillo C, Warshaw A L. Cystic pancreatic neoplasms. Ann Oncol 1999; 10 (Suppl 4):104-6.
5. Madura J A, Cummings O W, Wiebke E A, Broadie T A, Goulet R L Jr, Howard T J. Nonfunctioning islet cell tumors of the pancreas: a difficult diagnosis but one worth the effort. Am Surg. 1997; 63:573-7.
6. Zech C J, Helmberger T, Wichmann M W, Holzknecht N, Diebold J, Reiser M F. Large core biopsy of the pancreas under CT fluoroscopy control: results and complications. J Comput Assist Tomogr. 2002; 26:743-9.
7. Sparchez Z. Ultrasound-guided percutaneous pancreatic biopsy. Indications, performance and complications. Romanian Journal of Gastrointestinal and Liver Disease. 2002; 11(4):335-41.
8. J. L. Dulucq , P. Wintringer, C. Stabilini, T. Feryn, J. Perissat and A. Mahajna. Are major laparoscopic pancreatic resections worthwhile? A prospective study of 32 patients in a single institution. Surgical Endoscopy. 2005; 19:1028-1034.
9. David A. McClusky III, Lee J. Skandalakis, Gene L. Colborn, John E. Skandalakis. Harbinger or Hermit? Pancreatic Anatomy and Surgery through the Ages-Part 3. World Journal of Surgery. 2002; 26:1512-1242.
10. Furukawa H, Okada S, Saisho H, Ariyama J, Karasawa E, Nakaizumi A, Nakazawa S, Murakami K, Kakizoe T Clinicopathologic features of small pancreatic adenocarcinoma. A collective study. Cancer. 1996 Sep. 1; 78(5):986-90.
11. Mikael Hellström, Maria H. Svensson and Anders Lasson Extracolonic and Incidental Findings on CT Colonography (Virtual Colonoscopy) American Journal of Roentgenlogy. 2004; 182:631-638.
12. Riker A, Libutti S K, Bartlett D L. Advances in the early detection, diagnosis, and staging of pancreatic cancer. Surg Oncol. 1997; 6:157-69.
13. M Voss, P Hammel, G Molas, L Palazzo, A Dancour, D O'Tool, B Terris, C Degott, P Bernades, P Ruszniewski. Value of endoscopic ultrasound guided fine needle aspiration biopsy in the diagnosis of solid pancreatic masses Gut 2000; 46 244-249.
14. Bret P M, Nicole t V, Labadie M. Percutaneous fine-needle aspiration biopsy of the pancreas. Diagn Cytopathol. 1986; 2 221-7.
15. Bergenfeldt M, Genell S, Lindholm K, Ekberg 0, Aspelin P. Needle-tract seeding after percutaneous fine-needle biopsy of pancreatic carcinoma. Case report. Acta Chir Scand. 1988; 154 77-9.
16. Eoubeidi M A, Jhala D, Chhieng D C, Chen V K, Eltoum I, Vickers S, Mel Wilcox C, Jhala N. Yield of endoscopic ultrasound-guided fine-needle aspiration biopsy in patients with suspected pancreatic carcinoma. Cancer. 2003 Oct. 25; 99(5):285-92.
17. Afify A M, al-Khafaji B M, Kim B, Scheiman J M Endoscopic ultrasound-guided fine needle aspiration of the pancreas. Diagnostic utility and accuracy. Acta Cytol. 2003 May-June; 47(3):341-8.

It is intended that the foregoing description be only illustrative of the present invention and that the present invention be limited only by the hereinafter appended claims.

I claim:

1. A method for diagnosing pancreatic tumor masses in vivo in a patient which comprises:
(a) installing a laparoscopic port in an incision in the patient at about 10-12 mm at the right quadrant upper mid line of the patient;
(b) introducing a flexible gastroscope with a work channel with a length between about 8.6 and 12 mm through the port and into the peritoneal cavity of the patient with gas inflation of the peritoneum;
(c) introducing the gastroscope into the lesser sac in the peritoneal cavity and through the epiploic foramen adjacent to the pancreas;
(d) introducing biopsy forceps through the gastroscope work channel and removing a portion of a mass on the pancreas; and
(e) removing the endoscope from the patient and disinflating the peritoneal cavity, removing the laparoscopic port and closing the incision.

2. The method of claim 1 wherein the pancreas surgically enables repair of the pancreas.

3. A kit for performing the method of claim 1 or 2 comprising the gastroscope with the work channel and biopsy forceps adapted for the method which are disposable, the port, and means for closing the incision.
